(19) United States
(12) Patent Application Publication
(10) Pub. No.: US 2006/0172417 A1
(43) Pub. Date: Aug. 3, 2006

(54) Cell cultivation and breeding method

(76) Inventors: Jorg Rathenow, Eppstein (DE); Jurgen Kunstmann, Bad Soden (DE); Andreas Ban, Darmstadt (DE); Soheil Asgari, Wiesbaden (DE)

Correspondence Address: FROMMER LAWRENCE & HAUG, 745 FIFTH AVENUE- 10TH FL., NEW YORK, NY, 10151, US

(21) Appl. No.: 11/343,307

(22) Filed: Jan. 31, 2006

Related U.S. Application Data
(63) Continuation-in-part of application No. PCT/EP04/08642, filed on Aug. 2, 2004.
(63) Continuation-in-part of application No. PCT/EP04/00077, filed on Jan. 8, 2004.

(30) Foreign Application Priority Data
Jul. 31, 2003 (DE) 103 35 130.2

(51) Int. Cl. C12N 5/00

(52) U.S. Cl. 435/383

(57) ABSTRACT

The invention relates to a cell cultivation method, which may comprise the steps of preparing a carbon-based substrate with a layered structure, composed of at least two porous material layers, substantially superimposed and joined to each other, a gap which can be flowed through being formed between said layers, or of at least one porous material layer which is arranged or folded on itself, maintaining the shape thereof, such that a gap which can be flowed through is formed between at least two superimposed sections of the material layer. Said method then may comprise loading the substrate with a living and/or propagating biological material and contacting the loaded substrate with a liquid medium.

### INCORPORATION BY REFERENCE

This application is a continuation-in-part application of PCT Application No. PCT/EP2004/008642, filed Aug. 2, 2004 and designating the U.S., and published as WO 2005/012504 on Feb. 10, 2005, which claims benefit of German Application No. 103 35 130.2 filed Jul. 31, 2003 and PCT Application No. PCT/EP2004/00077, filed Jan. 8, 2004 and designating the U.S., and published as WO 2005/021462 on Mar. 10, 2005.

The foregoing applications, and all documents cited therein or during their prosecution (“appln cited documents”) and all documents cited or referenced in the appln cited documents, and all documents cited or referenced herein (“herein cited documents”), and all documents cited or referenced in herein cited documents, together with any manufacturer's instructions, descriptions, product specifications, and product sheets for any products mentioned herein or in any document incorporated by reference herein, are hereby incorporated herein by reference, and may be employed in the practice of the invention.

### FIELD OF THE INVENTION

The present invention relates to a method for culturing cells, which may comprise the steps of providing a carbon-based supporting body/substrate having a layered structure, may comprise at least two porous material layers that are essentially arranged on top of each other, between which a flow-throughable interspace exists; or at least one porous material layer that, while keeping its shape, is rolled up in itself or arranged in such a way that a flow-throughable interspace exists between at least two sections of the material layer that are on top of each other; and loading the supporting body with biological material which is living and/or capable of multiplication (viable) and contacting the loaded supporting body with a fluid medium.

### BACKGROUND OF THE INVENTION

In bioreactor process technology, it has become established practice in the meantime for substrate materials to be used to increase surface area. Systems available in the past have used mainly unordered structures in the form of granules, flocks, wafers or disks, capillaries, mesh, beads, etc., where the materials used are made mainly of ceramics or polymers. These systems usually have a great pressure drop and a limited surface area for volume yield. Here again, there is often a limitation on the size of the shaped bodies (pressure drop, weight, costs, packaging change, etc.), making it difficult to scale-up the process technically. In addition, polymers tend to undergo chemical or physical changes during use or sterilization. Furthermore, when there is an unordered packing, a uniform homogeneous nutrient supply and a reproducible filling cannot always be ensured. Dead space and preferred flow along the container walls lead to different metabolic conditions which can influence the product properties of sensitive proteins, such as their folding, for example.

Reactions on an industrial scale require a high throughput and are subject to economic factors. To be able to separate the metabolic products better from the cell mixture or for them to be reusable subsequently, the cells or cell cultures are immobilized on solid substrates. This yields a separation of the ambient medium from cells that are sensitive to shearing forces, for example. If a membrane is used as a wall of the shaped body, for example, and a cross geometry is used, for example, this yields the possibility of bringing gaseous metabolic products to the cells continuously without bubbles and/or enrich the desired metabolic products on one side of the membrane. This facilitates nutrient supply, exchange of metabolites and the measurement of process parameters and leads to a significant intensification of the process. Immobilization of cell cultures also permits continuous process management with a continuous supply and harvesting of product.

In addition, methods with immobilized cell cultures allow high cell densities so that comparatively high reaction rates and thus systems with smaller dimensions are possible and the yield can be increased drastically. With immobilized cell cultures from mammalian cell lines that have been genetically modified, e.g., for fermentation processes, higher reaction rates are achieved than with suspended cell cultures.

Especially in conjunction with “viable catalytic units,” it is important to note that the substrate is biocompatible, can be sterilized easily, offers a good adhesive base for the cell and allows the immobilization process to take place in a manner that is protective of the cells. Furthermore, the substrate must be adapted to the needs of the different cell cultures or cells. In this regard, the pore size and substrate composition play a role. There are already some methods of immobilizing cell cultures or cells.

For example, German Patent DE 693 11 134 relates to a bioreactor with immobilized lactic acid bacteria, where the bacteria are applied to a porous substrate. The substrate consists of a matrix of a plurality of loosely joined microparticles or microfibers. Cellulose or rayon and derivatives thereof are preferred. Agglomeration is preferably performed with polystyrene.

International Patent WO 01/19972 illustrates an immobilization process in which the cell cultures are combined with a polymer precursor and immobilized by subsequent crosslinking of the polymer.

Cell cultures may also be immobilized on open-pored “mineral” bulk materials as recited in International Patent WO 94/10095. Examples include expanded clay, expanded shale, lava, pumice, perlite and brick chippings.

Furthermore, International Patent WO 00/06711 relates to the immobilization of cell cultures or enzymes on diatomaceous earth as a substrate material.

European Patent 1270533 illustrates the use of crystalline oxide ceramic mixed with amorphous polyanionic intergranular phase in the form of granules and disks.

The methods mentioned above have certain disadvantages. The substrate matrices cannot be modified in any desired manner, for example, or the substrate material has a lower biocompatibility or the immobilization involves a high loss.

Immobilization of cell cultures in a polymer matrix by crosslinking a polymer precursor/cell mixture often results in many cell cultures dying during the polymer reaction, e.g., due to toxic reaction products or educts such as crosslinking agents. Furthermore, the crosslinked polymers are often swellable and therefore do not have dimensional stability and cause changes in flow conditions and therefore result in the mechanical stress in the cells.

Citation or identification of any document in this application is not an admission that such document is available as prior art to the present invention.

### SUMMARY OF THE INVENTION

An object of the present invention is to make available highly biocompatible, flexibly usable substrates that can be adapted to the particular application in a targeted manner for immobilizing viable (living) and/or propagable (capable of multiplication) biological material. Furthermore, another object of the present invention is to make available a cell culturing method which uses the aforementioned substrates. This method is preferably suitable for use on a laboratory scale and/or an industrial scale.

In the most general aspect, the present invention describes the use of a porous carbon-based body for immobilizing biological material for chemical and/or biological reactions. A cell culturing method is described for this purpose, using a porous supporting body/substrate material loaded with biological material. Suitable carbon-based supporting bodies/substrates loaded with biological material are also made available through this invention.

The solution to the problem according to this invention may include a method for culturing cell cultures on ordered carbon packings with a targeted flow through them with fluids, advantageously with a load specific pressure drop. The ordered packing of the inventive substrates yields on the one hand uniform flow conditions with the highest surface area to volume ratio for the purpose of nutrition for the cell cultures, while on the other hand also achieving an advantageous separation of the compartments into cell culture and medium. The substrates preferably may have channel-like structures between layers of material arranged one above the other or individual sections thereof. By varying the flow channel diameter and the channel wall thickness and/or the material layer thickness, optimum conditions in the substrate can be established in a flexible manner according to this invention for each application case. The flow ratios may be established, for example, by varying the channel geometry in the flow direction (e.g., corrugated channels), by variation in the diameter and variation in the surface properties of the carbon surface such as membrane properties, roughness, porosity, hydrophilicity, hydrophobicity, oleophilicity, oleophobicity, pH, impregnation with active ingredients and/or catalysts, etc. to adjust them to the required culture conditions.

Defined uniform supply conditions as well as substrate conditions of the substrate material are thus ensured within an intermediate area between two layers of material or sections thereof and/or within flow channels of the inventive substrate so that the cell cultures can always establish their optimum growth conditions at very high cell densities. The inventive substrates may also be installed easily in housings or containers and used in this form as cartridges either individually or with several combined together in industrial reactors or laboratory scale reactors for methods of cell culturing and breeding. According to this invention, an absolute reproducibility of the flow and substrate conditions for each cartridge produced in the same way is thus ensured, which represents an enormous simplification for approval proceedings in the pharmaceutical sector, for example.

The interaction of the inventive substrate and the cell cultures easily immobilized thereon, for example, with the medium can be accomplished in the inventive method here in several ways, e.g., by

flow of the medium through the substrates/cartridges by means of movement of the medium (e.g., by means of pistons, pressure, pumps, etc.)

movement of the substrate/cartridge in the medium,

movement of the substrate/cartridge with the medium through corresponding lines (e.g., by hydrostatic pressure).

Owing to the high chemical and physical stability of carbon, there is no problem with sterilization of the inventive substrate with conventional sterilization methods with which those skilled in the art are familiar in general. This permits, for example, optimum growth of cell cultures because the cells form colonies rapidly and adherently and/or adhesively on the carbon surface of the substrates and can thus be essentially separated from the ambient medium in the sense of forming a compartment. This makes it possible to achieve extremely high cell densities with a uniform and controllable nutrient supply and improved disposal of metabolites and harvesting of cell culture products.

According to the process aspect, the present invention therefore relate to a method for culturing cells may comprise the following steps:

a) providing a carbon based supporting body/substrate having a layered structure, which may comprise:
i) at least two porous material layers that are essentially arranged on top of each other, between which a flow-throughable interspace exists; or
ii) at least one porous material layer that, while keeping its shape, is rolled up in itself or arranged in such a way that a flow-throughable interspace exists between at least two sections of the material layer that are on top of each other; and

b) loading the supporting body with biological material which is living and/or capable of multiplication;

c) contacting the loaded supporting body with a fluid medium.

With regard to the product, the inventive solution to the above problems involves a porous carbon-based supporting body/substrate having a layered structure, which may comprise:

i) at least two porous material layers that are essentially arranged on top of each other, between which a flow-throughable space exists; or

ii) at least one porous material layer that, while keeping its shape, is rolled up in itself or arranged in such a way that a flow-throughable interspace exists between at least two sections of the material layer that are on top of each other;

which may comprise immobilized biological material which is living (viable) and/or capable of multiplication (propagable).

It is noted that in this disclosure and particularly in the claims and/or paragraphs, terms such as “comprises”, “comprised”, “comprising” and the like can have the meaning attributed to it in U.S. patent law; e.g., they can mean “includes”, “included”, “including”, and the like; and that terms such as “consisting essentially of” and “consists essentially of” have the meaning ascribed to them in U.S. patent law, e.g., they allow for elements not explicitly recited, but exclude elements that are found in the prior art or that affect a basic or novel characteristic of the invention.

These and other embodiments are disclosed or are obvious from and encompassed by, the following Detailed Description.

### BRIEF DESCRIPTION OF THE DRAWINGS

The following detailed description, given by way of example, but not intended to limit the invention solely to the specific embodiments described, may best be understood in conjunction with the accompanying drawings, in which:

FIGS. 1A-1C show schematically an embodiment of an inventive substrate having a layered structure.

FIGS. 2A-2B show schematically an embodiment of inventive cylindrical substrates having a circular oncoming flow area.

FIG. 3 shows schematically a device for implementing the inventive cell culturing method according to a preferred embodiment.

FIG. 4 shows schematically another device for implementing the inventive cell culturing method according to an alternative preferred embodiment.

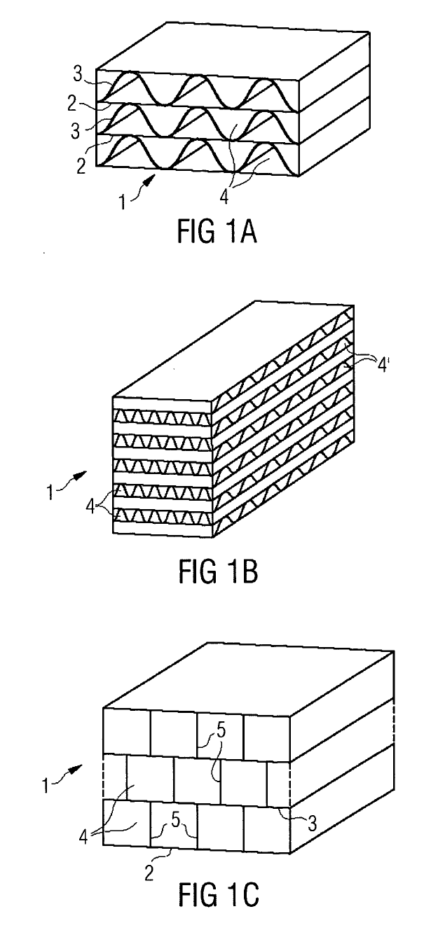

FIG. 1 shows embodiments of inventive supporting bodies/substrates having a layered structure. The substrate 1 shown in a perspective view in FIG. 1A comprises multiple alternating layers of material 2, 3 arranged one above the other, with a first material layer 2 being attached to an optionally structured, e.g., corrugated or pleated material layer 3 arranged above it so that an interspace is formed between the material layers 2 and 3, comprising a plurality of channels 4 through which the flow can pass in parallel. In the simplest space, the substrate of FIG. 1A may be imagined as a stack of corrugated cardboard. If the structured material layers are arranged alternately with an angular offset of 90°, for example, in relation to one another, the result is a substrate like that shown in FIG. 1B through which the flow can pass in an intersecting pattern in the channels 4, 4′. This substrate is essentially open at its end faces and has two possible directions of flow through the substrate offset in relation to one another due to the crosswise arrangement of the corrugated structure layers. As an alternative to structured material layers, two or more essentially flat material layers 2, 3 may also be arranged one above the other according to this invention, as shown in FIG. 1C, with two of these layers being joined together by spacer elements 5 so that a plurality of channels 4 through which the flow passes are provided in the interspaces between the material layers 2, 3.

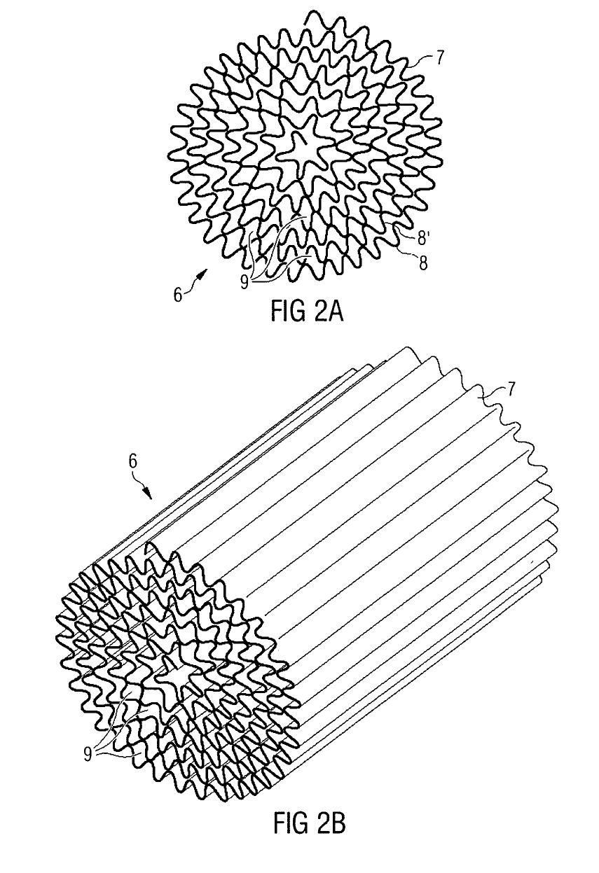

FIG. 2 shows another embodiment of the supporting body/substrate of the present invention. The top view of the cylindrical substrate 6 in FIG. 2A shows a corrugated material layer 7 rolled up in a spiral shape. This coiling results in a plurality of areas by means of which another section 8′ on the material layer 7 rests on a section 8 of the material layer in the next winding so that intermediate channels 9 are formed between the sections 8 and 8′. As shown in FIG. 2B, the substrate 6 has a cylindrical structure due to the fact that a flat sheet having a corrugated structure is coiled up or rolled up. Corresponding substrates can be rolled up, e.g., by rolling up corrugated paperboard to form a cylindrical shaped body. By carbonizing the resulting corrugated cardboard material, cylindrical shaped bodies 6 can be formed, having a plurality of channels 9 passing through them in the direction of the height of the cylinder. This yields a cylindrical substrate 7 with a circular end face through which flow can pass essentially unidirectionally (FIG. 2A).

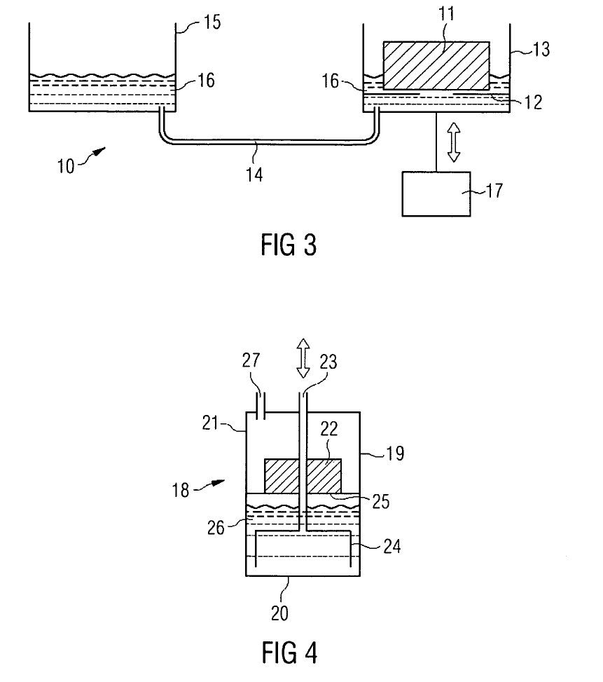

FIG. 3 shows a schematic diagram of a preferred embodiment of a device and/or a reactor 10 for implementing the cell culturing method according to the present invention. A supporting body/substrate 11, e.g., in the form of a cylinder as illustrated in FIG. 2 or a block as illustrated in FIG. 1 rests on a suitable holder 12, e.g., a perforated plate in a reactor vessel 13. This reactor vessel 13 is connected via an equalizing line 14 to an equalizing and storage container 15 which contains the fluid medium 16, e.g., a culture medium. The reactor vessel 13 is movable up and down with respect to the equalizing container 15 by means of a suitable device 17. In the downward movement of the reactor vessel 13, medium 16 flows out of the storage container 15 through the line 4 into the reactor vessel 13 so that the substrate 11 is partially or completely immersed in the culture medium, depending on the vertical alignment of the reactor vessel 13 with respect to the fluid level in the storage container 15. By regularly moving the reactor vessel 13 up and down, the substrate 11 is cyclically immersed in the culture medium 16, so that the substrate 11 has medium 16 flowing through it. The reactor vessel 13 may optionally be sealed airtight and the gas space above the medium in the reactor vessel 13 may optionally be filled with inert gas, in which optionally a pressure equalizing device may be provided. By moving the reactor vessel up and down, the medium 16 is moved into the flow channels of the substrate 11 in such way as to permit a uniform supply of moisture, nutrients or the like to microorganisms or cells or cell tissues. At the same time, metabolites created by microorganisms, cells or other biological material immobilized on the substrate 11 can be carried away from the substrate 11 by the medium 16. These metabolites accumulate in the medium 16 and can be removed from it via the equalizing line 14 or the storage container 15 either continuously or discontinuously, e.g., by extraction or similar separation methods.

FIG. 4 shows another embodiment of a device 18 for performing the inventive cell culturing method which works by the alternating pressure principle. An inventive supporting body/substrate 22, e.g., in the form of a cylinder section of the substrate as shown in FIG. 2 or in block form as shown in FIG. 1 is situated in reactor vessel 19 with two chambers 20, 21 arranged one above the other. This substrate 22 has a radial borehole through which compressed air can be introduced through a differential pressure input 23 into a displacement space 24 situated in the lower reactor chamber 20. The two chambers 20, 21 of the reactor vessel 19 are separated from one another by a permeable reactor partition 25, which may be a perforated bottom, for example, on which the substrate 22 rests. For operation of the reactor, the lower reactor chamber 20 is filled with fluid medium 26, e.g., a liquid culture medium for microorganisms or cells, so that the liquid level remains below the reactor partition 25. If compressed air is introduced into the displacement room 24 through the differential pressure input 23, then part of the liquid culture medium 26 is displaced into the lower reactor chamber 20 according to the immersion bell principle and is forced upward through the reactor partition 25, so that the substrate 22 comes into contact with the liquid culture medium 26. The excess pressure prevailing in the upper reactor chamber is released through a pressure equalizing opening 27 in the upper reactor chamber 21. By regularly or irregularly putting the lower reactor chamber 20 under pressure and then releasing the pressure through the differential pressure input 23 into the displacement space 24, the substrate 22 is flushed with liquid culture medium 26. In doing so, the substrate 22 may be immersed completely or partially into the medium 26.

### DETAILED DESCRIPTION

Inventive carbon-based supporting bodies/substrates have an excellent biocompatibility when used as supporting body/substrate materials for cell cultures or cells; they are free of toxic emissions, have dimensional stability and are extremely versatile with regard to their design such as pore size, internal structure and external shape. Furthermore, the inventive porous bodies are easily sterilized and offer a good adhesive substrate for microorganisms, cell cultures and cells as well as viable and/or propagable biological material in general. Because of these properties, these porous bodies based on carbon can be tailored to meet the requirements of a variety of applications. The porous substrates preferably consist primarily of amorphous and/or pyrolytic and/or vitreous carbon, preferably selected from activated carbon, sintered activated carbon, amorphous crystalline or partially crystalline carbon, graphite, pyrolytic carbonaceous material, carbon fibers or carbides, carbonitrides, oxycarbides or oxycarbonitrides of metals or nonmetals as well as mixtures thereof or similar carbon-based material. The porous supporting bodies/substrates of the present invention are especially preferably pyrolytic material consisting essentially of carbon.

The supporting bodies/substrates are optionally especially preferably produced by pyrolysis/carbonization of starting materials which are converted under a high temperature in a oxygen-free atmosphere to the aforementioned carbon-based materials. Suitable starting materials for carbonization of the inventive substrates include for example, polymers, polymer films, paper, impregnated or coated paper, wovens, nonwovens, coated ceramic disks, cotton batting, batting rods, batting pellets, cellulose materials or, for example, legumes such as peanuts, lentils, beans and the like or nuts, dried fruit or the like as well as greenware produced on the basis of these materials.

The term “carbon-based” as used in the context of the present invention is understood to refer to all materials having a carbon content (prior to any modification with metals) of more than about 1 wt %, more than about 10 wt %, more than about 20 wt %, more than about 30 wt %, more than about 40 wt %, in particular more than about 50 wt %, more than about 55 wt %, preferably more than about 60 wt %, more than about 65 wt %, especially preferably more than about 70 wt %, more than about 75 wt %, more than about 80 wt %, more than about 85 wt % and especially more than about 90 wt %. In especially preferred embodiments, the inventive carbon-based supporting bodies/substrates have a carbon content between about 95 and about 100 wt %, in particular about 95 to about 99 wt %.

It is preferably for the supporting body/substrate to have a plurality of layers of material arranged one above the other, each forming an interspace through which the flow can pass. Preferably each interspace includes channel-like structures, e.g., a plurality of channels arranged essentially in parallel, intersecting or in a network. The channel-like structures may be arranged a distance apart from one another due to a plurality of spacer elements provided on the layers of substrate material so that the distance is ensured. The channels, i.e., channel-like structures, preferably have an average channel diameter in the range of approximately one nanometer to approximately one meter, from approximately one nanometer to approximately one hundred centimeters, in particular from approximately one nanometer to approximately ten centimeters, from approximately one nanometer to approximately ten millimeters, preferably about ten nanometers to about ten millimeters and most especially about fifty nanometers to about one millimeter. The distance between two adjacent layers of material will have essentially identical dimensions.

The inventive supporting body/substrate is especially preferably designed so that the channels between a first and a second material layer and the channels in an adjacent layer between the second and third material layers are arranged in essentially the same direction so that on the whole, the substrate has channel layers through which a flow can pass in a preferred direction. Alternatively, the substrate may also be designed so that it has channel layers alternately offset by an angle in relation to one another between a first and a second layer of material are arranged with an offset at an angle of more than about 0° up to about 90°, more than about 10° up to about 90°, more than about 20° up to about 90°, preferably about 30° to about 90°, about 35° up to about 90°, about 40° up to about 90° and especially preferably about 45° to about 90° with respect to the channels in an adjacent layer between the second material layer and the third material layer.

The channels, i.e., channel-like structures in the inventive substrate are essentially open at both ends of the channels so that the inventive body on the whole has a type of sandwich structure, i.e., a layered design of alternating layers of porous material and interspaces, preferably channel layers through which the flow can pass between them. The channels, i.e., channel-like structures may have a linear extent in their longitudinal direction according to this invention or they may have a corrugated, meandering or zigzag pattern and may run in parallel or intersecting one another within an interspace between two layers of material.

The outer form and dimensions of the inventive supporting body/substrate can be selected and adapted according to the particular intended application. The supporting body/substrate may have an external form which is selected, for example, from elongated shapes such as cylindrical, polygonal column shapes such as a triangular column shaped or a bar shape or may be in the form of a sheet or a polygonal shape, e.g., quadratic, cuboid, tetrahedral, pyramidal, octahedral, dodeca-hedral, icosahedral, rhomboid, prismatic or spherical, hollow spherical or cylindrical, lenticular or disk-shaped or ring-shaped.

Inventive substrates may be dimensioned in a suitable manner in relation to the intended application, e.g., with a supporting body/substrate volume in the range from about 1 mm³, about 10 mm³, about 1 cm³, preferably approximately about 10 cm³ to about 1 m³. In cases where this is desirable, the substrates may also be dimensioned to be much larger or even on a smaller micro-dimension, the present invention is not limited to certain dimensions of the substrate. The substrate may have a longest outer dimension in the range from approximately about one nanometer to about one thousand meters, about ten nanometers to about five hundred meters, about one-tenth centimeter to about one hundred meters, preferably approximately about five-tenths centimeter to about fifty meters, about five-tenths centimeter to about ten meters, about one centimeter to about ten meters, especially preferably approximately about one centimeter to about five meters.

To do so, a corrugated layer of material, for example, may be rolled up in a spiral pattern to form a cylindrical body. Such substrates are designed so that one layer of material, optionally corrugated, embossed or otherwise structured in a manner that retains its shape is arranged in a spiral, forming an intermediate area between at least two portions of the material layer arranged one above the other so that the flow can pass through the intermediate area, preferably having a plurality of channel-like structures and/or channels.

Several layers of material arranged one above the other can be shaped to form such cylindrical supporting bodies/substrates by rolling them up.

The porous layers of material and/or the channel walls and/or spacer elements between the layers of material of the inventive supporting bodies/substrates may have an average pore size in the range of approximately one nanometer to ten centimeters, approximately one nanometer to ten millimetres, preferably about ten nanometers to about ten millimeters, preferably about ten nanometers to about one millimeter and especially preferably about fifty nanometers to about one millimeter. The porous layers of material are optionally semipermeable and generally have a thickness of between about three Angstrom and about ten centimeters, between about one nanometer and about ten centimeters, between about one nanometer and about one centimeter, between about one nanometer and about 100 millimeters, between about one nanometer and about 10 millimeters, between about one nanometer and about one millimetre, preferably from about one nanometer to about one hundred micrometers and most preferably from about ten nanometers to about ten micrometers. The average pore diameter of the porous layers, optionally semipermeable, is between about one-tenth Angstrom and about one millimeter, between about one-tenth Angstrom and about one hundred micrometers, preferably about one Angstrom to about one hundred micrometers, about three Angstroms to about one hundred micrometers and most preferably from about three Angstroms to about ten micrometers.

In a preferred embodiment of the supporting body/substrate of the present invention, the material layers of the supporting body/substrate are structured on one or both sides, preferably on both sides. The preferred structuring of the material layers consists of the shape of an embossed groove pattern or a pattern otherwise introduced with grooves and/or channel-like recesses arranged essentially equidistant from one another over the entire surface of the material layers. The groove patterns may run in parallel with respect to the outer edges of the material layers or may be arranged at any angle thereto, or may have a zigzag pattern or a corrugated pattern. Furthermore the material layers, if structured on both sides may have identical groove patterns on both sides or may have different groove patterns. It is preferably for the porous material layers to be structured so they are uniformly complementary on the two sides, i.e., the groove recesses on one side of the material correspond to a corresponding elevation in the profile on the other side of the material layer. The material layers are preferably arranged in the substrates so that the groove patterns of two neighboring material layers run essentially parallel to one another.

Furthermore, the material layers may be arranged in such a way that the groove pattern of two neighboring material layers intersect at an angle so that when the material layers are stacked one above the other, the result is a plurality of points of contact between the neighboring material layers at the points of intersecting elevated edges of the groove structure of neighboring material layers. This yields substrates having a definitely increased mechanical stability owing to the connection at many point according to the points of contact of the intersecting groove pattern. The groove structures are selected in particular in such a way that when two layers of material are arranged one above the other, a channel-like or network structure is formed in the intermediate areas between two neighboring layers of material, corresponding to a plurality of channels or tubes and ensuring a suitable flow resistance in the supporting body/substrate, preferably the lowest possible flow resistance. Those skilled in the art will know how to select the groove patterns and dimensions appropriately. In the inventive supporting body/substrate, the conventional groove structures in embossed layers of material lead to channel-like structures and/or tubular structures in intermediate spaces whose cross-sectional area can be adapted to the particular intended purpose.

As an alternative to embossing of grooves or channels, the material layers may also have preformed corrugation or they may have accordion pleating. When a plurality of such material layers are arranged flatly one on top of the other, the result is a honeycomb structure as seen from the end face of the supporting body/substrate, running as channel structures in the direction of the plane of the layers of material. When such preformed material layers are rolled up, the result is cylindrical substrates whose cross section has a plurality of channels arranged in a spiral, extending along the longitudinal dimension of the cylinder. Such cylinders/disks are essentially open at the cross-sectional faces on both ends.

In addition, spacer elements may also be provided and/or introduced alternatively or additionally between the material layers. Corresponding spacer elements serve to ensure sufficiently large interspaces between the material layers in which the channels run and which ensure a suitable low flow resistance of the module. Corresponding spacer elements may be porous, open-pored flat sheeting in the form of intermediate layers, network structures or spacers arranged centrally or at the edges of the material layers, which then ensure a certain minimum distance between the material layers.

The inventive supporting bodies/substrates have intermediate layers and/or channels and/or channel layers which are essentially open at both ends of the channels and/or layers. Inventive substrates are not sealed or closed with respect to fluids on the ends and edges of the material layers and/or at the entrances or outlets to the channels.

The spacing of the material layers with respect to one another is especially preferably ensured by the fact that a plurality of points of contact between the neighboring layers of material is obtained at the points of intersecting elevated edges of the structures due to suitably dimensioned groove embossing, pleating or corrugation and intersection of the groove pattern, the pleat pattern or the corrugation pattern of two neighboring layers of material in a certain angle. This ensures that interspaces in the form of a plurality of channel-like structures are formed along the recesses in the material layers. Similarly, this may also be accomplished through alternating folds or corrugations in the material layer of different widths.

Furthermore, the material layers may also be arranged a distance apart so that groove embossing or pleating and/or corrugation of different depths in alternation is provided on the material layers, leading to elevations of individual groove edges of different heights so that the number of points of contact between the neighboring material layers at the points of intersecting edges of the grooves structures, the corrugated structure or pleated structures is reduced on the whole in a suitable manner in comparison with the total number of groove edges available. By joining the material layers at these points, an adequate strength of the supporting body/substrate is ensured and a good flow resistance is ensured.

It is especially preferable for a module structure to be used as the porous supporting body/substrate, this structure being created by carbonization of an optionally structured, embossed, pretreated and pleated sheeting based on fiber, paper, textile, or polymer material. Supporting bodies/substrates according to this invention accordingly consists of a carbon-based material, optionally also corresponding to a carbon composite material produced by pyrolysis of carbonaceous starting materials and essentially a type of carbon ceramic and/or carbon-based ceramic. Such materials can be produced, for example, starting from paper-like starting materials by pyrolysis and/or carbonization at high temperatures. Corresponding production processes, in particular also those for carbon composite materials, are described in International Patent Application WO 01/80981, in particular page 14, line 10 through page 18, line 14 there and can be applied in the present case. The inventive carbon-based substrates may also be produced according to the method described in International Patent Application WO 02/32558, in particular page 6, line 5 through page 24, line 9 there. The disclosure of these International Patent Applications is herewith included completely by citation.

Inventive substrates can also be obtained by pyrolysis of suitably prefabricated polymer films and/or three-dimensionally arranged or folded polymer film packets, as described in German Patent DE 103 22 182, the disclosure content of which is herewith completely included through this reference.

Especially preferred embodiments of the inventive supporting body/substrate can be produced in particularly by carbonization of corrugated paperboard according to pyrolysis methods described in the aforementioned patent applications, whereby the corrugated paperboard layers are suitably secured on one prior to carbonization, resulting in an open body through which a flow can pass.

In addition, preferred substrates are also obtained in cylindrical form by rolling up or coiling layers of paper or polymer film or stacks of paper or polymer film to form cylindrical bodies, tubes or rods arranged in parallel or for cross flow and their subsequent pyrolysis according to the aforementioned methods of the state of the art. These “coiled bodies” in the simplest case include a grooved, embossed, pleated or corrugated porous material layer that is coiled up to form a cylinder by rolling up this sheet-like precursor and then is carbonized after being rolled up. The resulting cylindrical supporting body/substrate includes a layer of porous material rolled up in a spiral or like a worm gear in cross section, the interspaces and/or channels extending essentially in the direction of the height of the cylinder between the windings of the supporting body/substrate, with the cross section serving as the oncoming flow area having the lowest flow resistance. Similarly, two or more material layer precursors arranged one above the other can also be rolled up and then carbonized to form the supporting body/substrate. At least two material layers arranged in alternation one above the other, one being a corrugated layer and the other being essentially flat (cover layer) are also especially preferred; this prevents the corrugations and/or grooves from slipping into one another when rolled up to form a cylinder and therefore the interspaces forming a channel-like structure are kept open. Example 1 below describes such cylindrical shaped bodies.

The inventive supporting bodies/substrates may optionally be modified to adapt the physical and/or chemical-biological properties to the intended application. Carbon-based materials are basically highly biocompatible substances which form an ideal substrate for cells, microorganisms or tissue. Inventive substrates may be modified on their internal and/or external surfaces to be at least partially hydrophilic, hydrophobic, oleophilic or oleophobic, e.g., by fluoridation, parylenation, by coating or impregnating the supporting body/substrate with substances that promote microbial growth, culture media, polymers, etc.

The properties of the supporting body/substrate may especially preferably be modified with other substances selected from organic and inorganic substances or compounds. The preferred substances are compounds of iron, cobalt, copper, zinc, manganese, potassium, magnesium, calcium, sulfur or phosphorus. The incorporation of these additional compounds may be used, for example, to promote the growth of certain microorganisms or cells on the substrate. Furthermore, impregnation or coating of the supporting body/substrate with carbohydrates, lipids, purines, pyromidines, pyrimidines, vitamins, proteins, growth factors, amino acids and/or sulfur sources or nitrogen sources are also suitable in promoting growth. Furthermore, the following substances may be used to stimulate cell growth: bisphosphonates (e.g., risedronates, pamidronates, ibandronates, zoledronic acid, clodronic acid, etidronic acid, alendronic acid, tiludronic acid), fluoride (disodium fluorophosphate, sodium fluoride); calcitonin, dihydrotachystyrene as well as all growth factors and cytokins (epidermal growth factor (EGF), platelet-derived growth factor (PDGF), fibroblast growth factors (FGFs), transforming growth factors β (TGFs-β), trans-forming growth factor α (TGF-α), erythropoietin (Epo), insulin-like growth factor I (IGF-I), insulin-like growth factor II (IGF-II), interleukin 1 (IL-1), interleukin 2 (IL-2), interleukin 6 (IL-6), interleukin 8 (IL-8), tumor necrosis factor α (TNF-α), tumor necrosis factor β (TNF-β), interferon γ (INF-γ), monocyte chemo-tactic protein, fibroblast stimulating factor I, histamine, fibrin or fibrinogen, endothelin 1, angiotensin II, collagens, bromocriptine, methysergide, methotrexate, carbon tetrachloride, thioacetamide, ethanol).

The flow conditions in the supporting body/substrate can be adjusted, for example, to the required culture conditions by varying the geometry of the interspace or the channels in the direction of flow (e.g., corrugated channels), by varying the diameter and optionally also the surface properties of the carbon surface such as the membrane properties, roughness, porosity, hydrophilicity, hydrophobicity, oleophilicity, oleophobicity, pH, impregnation with active ingredients and/or catalysts, etc.

By the method according to this invention, the supporting body/substrate is loaded with viable and/or propagable biological material. The biological material preferably includes single-cell or multi-cell microorganisms, fingi, spores, viruses, plant cells, cells culture or tissue or animal or human cells, cell cultures or tissue or mixtures thereof. The loading preferably leads to extensive immobilization of the biological material.

The loading is preferably performed with tissue-forming or non-tissue-forming mammalian cells, algae, bacteria, in particular genetically modified bacteria producing active ingredients, primary cell cultures such as eukaryotic tissue, e.g., bone, cartilage, liver, kidney as well as exogenous, allogeneic, syngeneic or autologous cells and cell types and optionally also genetically modified cell lines and in particular also nerve tissue.

The biological method can be applied to the supporting body/substrate by conventional methods. Examples include immersion of the supporting body/substrate in a solution/suspension of the cell material, spraying the supporting body/substrate with cell material solution or suspension, inoculating a fluid medium in contact with the supporting body/substrate and the like. An incubation time is optionally necessary after loading to allow the immobilized biological material to completely permeate the supporting body/substrate.

The carbon-based substrates are suitable in particular for immobilizing and propagating microorganisms of all types and tissue cultures, especially cell tissues. In these processes, the microorganisms and/or cell cultures form colonies on the substrates and can be supplied with liquid or gaseous nutrients through the flow-through intermediate layers and/or flow channels in the intermediate layers, while metabolites can be removed easily with a fluid flow passing through the supporting body/substrate. Furthermore, the microorganisms and cells largely immobilized on the supporting body/substrate can be protected from being discharged and from possible harmful environmental influences such as mechanical stresses.

Furthermore, it is possible according to this invention to immerse several substrates having different microorganisms, cell cultures or tissue cultures into a reaction mixture containing, for example, a reaction medium and optionally the educts and thus allow the reaction medium to pass through them without resulting in mixing of microorganisms, cell or tissue cultures that are largely immobilized on the substrates.

The corresponding supporting bodies/substrates, optionally installed in suitable housings to form cartridge systems which are loaded with different microorganisms or optionally different cell cultures, may be immersed in a single culture medium for the sake of reproduction or active ingredient production and may be removed from the culture medium after a certain period of time as individual cartridges for harvesting and open for this purpose or the products may be removed continuously. The substrates or the housings and/or cartridges containing the substrates may optionally also be designed so that they must be destroyed to release the active ingredient or they may be opened of closed in a reversible procedure. The cartridges are preferably designed to be reversibly opened and reclosed.

According to this invention, the supporting bodies/substrates may optionally be arranged in a suitable housing or in or on a suitable container selected from reactors for chemical or biological reactors, e.g., flasks, bottles, in particular cell culture flasks, roller bottles, spinner bottles, culture tubes, cell culture chambers, cell culture dishes, culture plates, pipette caps, snap cover dishes, cryotubes, agitated reactors, fixed bed reactors, tubular reactors or the like.

Before, during or after loading with the biological material, the supporting body/substrate is brought in contact with a fluid medium. The fluid medium may optionally be a different medium before loading than after loading. The term “fluid medium” includes any fluid, gaseous, solid or liquid, such as water, organic solvents, inorganic solvents, supercritical gases, conventional substrate gases, solutions or suspensions of solid or gaseous substances, emulsions and the like. The medium is preferably selected from liquids or gases, solvents, water, gaseous or liquid or solid reaction educts and/or products, liquid culture media for enzymes, cells and tissues, mixtures thereof and the like.

Examples of liquid culture media include, for example, RPMI 1640 from Cell Concepts, PFHM II, hybridoma SFM and/or CD hybridoma from GIBCO, etc. These may be used with or without serum, e.g., fetal bovine serum medium with or without amino acids such as L-glutamine. The fluid medium may also be mixed with biological material, e.g., for inoculating the supporting body/substrate.

The contact may be accomplished by complete or partial immersion of the supporting body/substrate or the housing/container holding it into the fluid medium. The substrates may also be secured in suitable reactors so that fluid medium can flow through them. An important criterion here is the wettability and removability of any enclosed air bubbles from the substrate material. Evacuation, degassing and/or flushing operations may be necessary here and may be used as needed.

After a first contact between the supporting body/substrate and a fluid medium, the biological material is preferably then added, i.e., usually in liquid form, e.g., as a solution, suspension, emulsion or the like, especially preferably in the fluid medium itself, usually under sterile conditions. With the inventive substrates, there is usually a clarification of the medium environment which has a certain opacity due to the cells, usually clarifying after a few hours, often after approximately two hours.

The supporting body/substrate is preferably immersed in a solution, emulsion or suspension containing the biological material for a period for time from 1 second up to 1000 days or may be inoculated with it, optionally under sterile conditions, to give the material an opportunity to diffuse into the porous body and form colonies there. The inoculation may also be performed by spray methods or the like.

The fluid medium, e.g., a culture medium, may be moved or agitated to ensure the most homogeneous possible vital environment and supply of nutrients to the microorganisms. This may be accomplished through various methods as indicated above, e.g., by moving the supporting body/substrate in the medium or moving the medium through the supporting body/substrate. This is usually done for a sufficient period of time to permit growth, reproduction or adequate metabolic activity of the biological material.

Then the metabolites, i.e., the proliferated cells, are harvested. The fixed culturing on the supporting body/substrate surface here is a desired simplification because the cells and the ambient medium can be easily separated from one another in this way. The cells adhere well to the supporting body/substrate and can be removed by suitable means after washing off the medium, optionally flushing it out, with suitable means.

After harvesting the metabolic products, e.g., by extraction from the medium, the supporting body/substrate may, if desired or necessary, be purified, sterilized and reused for reloading with the same or different biological material. For subsequent reuse of the loaded substrates, they may also be preserved by cryopreservation together with the biological material.

The inventive method is preferably implemented with one (or more) substrates which is/are introduced into a suitable housing, container or reactor or reactor system before or after loading with biological material. The substrate is preferably brought in contact with the fluid medium in the housing, container or reactor or reactor system by at least partially filling the housing container or reactor and/or reactor system.

The contact with the medium preferably takes place in one embodiment in such a way that the substrate is continuously or discontinuously brought into motion with the medium in the housing, container or reactor and/or reactor system. To do so, the container is usually connected to a storage container filled with medium via feed mechanisms and, if necessary, additional removal mechanisms are provided to carry the medium continuously or discontinuously into and through the container. As an alternative, the supporting body/substrate may also be moved by means of suitable devices in a housing, container or reactor and/or reactor system partially or entirely filled with the fluid medium by means of suitable devices.

Furthermore, the supporting body/substrate may be continuously or discontinuously, optionally entirely or partially immersed in a housing, container or reactor and/or reactor system so that a fluid medium can flow through it. In doing so, the flow of fluid medium through the supporting body/substrate may be accomplished by moving the supporting body/substrate in the medium. Alternatively, the flow of fluid medium through the supporting body/substrate may be accomplished by moving the medium in the supporting body/substrate, e.g., by means of suitable agitator mechanisms, pump system, pneumatic medium lifting devices and the like. After loading the supporting body/substrate with the biological material, nutrients are preferably added and/or metabolic products are preferably removed continuously or discontinuously along with the biological material.

In the method according to this invention, the supporting body/substrate is loaded and/or inoculated with a suitable amount of biological material corresponding to the intended purpose. The material is preferably loaded and/or inoculated in such a way that the supporting body/substrate contains between about 10⁻⁵ wt % and about 99 wt %, between about 10⁻⁴ and about 90 wt %, between about 10⁻³ and about 85 wt %, preferably between about 10⁻² wt % and about 80 wt %, between about 10⁻¹ and about 70 wt %, between about 10⁻¹ and about 60 wt %, of at least preferably between about 1 and about 50 wt % cells, based on the total weight of the loaded supporting body/substrate. The supporting body/substrate especially preferably contains cell cultures in the amount of up to 10⁶ times its only weight as well as having a cell density of 1 to 10²³ cells per mL of supporting body/substrate volume.

The inventive method is especially suitable for culturing and optionally reproducing nerve tissue. It is especially advantageous here that the inventive carbon-based substrates are also especially adaptable and suitable due to the ease with which the conductivity of the bodies is adjusted and the application of pulsed currents to culture nerve tissue.

According to this invention, the substrates may be used of culturing in conventional bioreactor systems, e.g., passive systems without continuous regulating techniques such as tissue plates, tissue bottles, roller bottles as well as active systems with input of gas and automatic adjustment of parameters (acidity, temperature), i.e., reactor systems in the broadest sense with measurement and control technology.

Furthermore, the inventive vehicle bodies can also be operated as a reactor system by providing suitable equipment, e.g., connections for perfusion with culture media and gas exchange, in particular also including modular designs in corresponding series reactor system and tissue cultures.

According to this invention it is preferably to perform the cell culturing method with a reactor and/or a reactor system comprising at least one supporting body/substrate as described above, whereby the reactor and/or the reactor system is selected from flasks, bottles, especially cell culture bottles, roller bottles, spinner bottles, culture tubes, cell culture chambers, cell culture dishes, culture dishes, cryotubes, agitated reactors, fixed bed reactors, tubular reactors. Roller bottles comprising an inventive supporting body/substrate or cartridges comprising an inventive supporting body/substrate in a housing are especially preferred.

In addition, the inventive substrates may also be modified appropriately for promoting organogenesis, e.g., with proteoglycans, collagens, tissue salts, e.g., hydroxyl apatite, etc., especially with the above mentioned biodegradable and/or absorbable polymers. The inventive substrates are furthermore preferably also modified by impregnation and/or adsorption of growth factors, cytokines, interferons and/or addition factors. Examples of suitable growth factors include PDGF, EGF, TGF-α, GFG, NGF, erythropoietin, TGF-β, IGF-I and IGF-II. Suitable cytokines include, for example, IL-1-α and IL-1-β, IL-2, IL-3, IL-4, IL-5, Il-6, IL-7, IL-8, IL-9, IL-10, IL-11, IL-12, IL-13. Suitable interferons include, for example, INF-α and INF-β, INF-γ. Examples of suitable adhesion factors include fibronectin, laminin, vitronectin, fetuin, poly-D-lysine and the like.

The cell density of the inventive supporting bodies/substrates may be in the range from about 1 to 10²³ cells per mL volume, about 1 to 10²² cells per mL volume, about 1 to 10²¹ cells per mL volume, about 1 to 10²⁰ cells per mL volume, about 1 to 10¹⁹ cells per mL volume, about 1 to 10¹⁸ cells per mL volume, about 1 to 10¹⁷ cells per mL volume, about 1 to 10¹⁶ cells per mL volume, about 1 to 10¹⁵ cells per mL volume, about 1 to 10¹⁴ cells per mL volume, about 1 to 10¹³ cells per mL volume, about 1 to 10¹² cells per mL volume, about 1 to 10¹¹ cells per mL volume, about 1 to 10¹⁰ cells per mL volume, about 1 to 10⁹ cells per mL volume, in particular reactor volume, preferably up to about 10², about 10³, about 10⁴, preferably about 10⁵, about 10⁶, about 10⁷, about 10⁸ especially up to about 10⁹ cells per mL.

The reactors and/or reactor systems may be operated continuously or in batches. The inventive supporting body/substrate may have a semipermeable separation layer in these systems. Substrates without a semipermeable separation layer may be installed into a container in the reactor, preferably containing a semipermeable separation layer. In such a case the container is preferably designed so that the mass exchange between the fluid medium in the reactor and the interior of the container is controlled through the semipermeable separation layer. The semipermeable separation layer may have the same separation properties as the semipermeable separation layer in contact with the outside surface of the porous supporting body/substrate.

For the use of substrates having a semipermeable separation layer or substrates which are in a container having a semipermeable separation layer which allows mass exchange only with respect to the educts and the reaction medium, agitated vessel reactors operated in batches are preferred, likewise without any separation layer for inventive substrates. These agitated vessel reactors are usually equipped with an agitator and optionally with a continuous educt feed mechanism. The substrate(s) is/are optionally immersed into the fluid medium inside a container which optionally has a semipermeable separation layer. If comparatively small supporting bodies/substrates are used, they are preferably accommodated in a container or housing when immersed into the medium. The container allows contact with the medium, optionally through a semipermeable separation layer, but it prevents uncontrolled distribution of the substrates in the reactor.

The flow in the reaction space is preferably turbulent and the laminar boundary film is preferably as thin as possible. To maintain a gradient, a good convection effect is necessary. Educts must always be supplied in sufficient quantity. Those skilled in the art will recognize that measures leading to a good and thorough and good convection are also suitable for the present invention.

Those skilled in the art will be aware that the mass transport becomes faster with an increase in turbulence (increasing Re number) due to the reduction in the diffusion pathway. The shorter the diffusion pathways and the greater the concentration gradient, the more rapid is the mass transport between the interior space and the exterior space. Those skilled in the art will be aware that the rate of most reactions is determined by the mass transport and not by the reaction rate and thus the reaction rate depends directly on the mass transport. Only in exceptional cases is the reaction rate itself slower than the mass transport, so the reaction rate is limited by the actual reaction and not by the mass transport.

Alternatively, a continuous process management may also be used. A continuous process management brings the advantage that educts can be supplied continuously or discontinuously with the fluid medium and products can be removed continuously or discontinuously. For this embodiment, supporting bodies/substrates without a semipermeable separation layer are preferred. As an alternative to substrates having a semipermeable separation layer, substrates that do not have a semipermeable separation layer are immobilized in a container or housing when introduced into the reactor having a semipermeable separation layer may also be used. Preferred reactors include continuously operated stirred vessel reactors, tubular reactors and optionally also fluidized bed reactors.

The reactor dwell time will be vary, depending on the reaction, and will depend on the rate of the biological reaction. Those skilled in the art will adjust the dwell time according to the particular reaction. The educt stream can preferably be carried in circulation, whereby suitable measurement and control equipment is provided to control, for example, the temperature, pH, nutrient concentration or educt concentration in the medium. Products can be removed from the circulating stream either continuous or discontinuously.

The inventive supporting bodies/substrates may either be anchored fixedly in the agitated vessel or tubular reactor or they may float loosely in the medium or they may be contained in a container or housing that is immersed in the reaction medium. If they bodies float freely in the medium, means must e provided at the reactor outlet to ensure that these bodies cannot leave the reactor. For example, screens may be mounted at the outlet. The inventive supporting bodies/substrates are preferably arranged in a porous container or housing, which is optionally provided with a semipermeable separation layer, for immersion in the reaction mixture. This embodiment also offers the advantage that the substrates can be removed easily when the agitated vessel is needed for other reactions or if replenishing is necessary.

In another embodiment of this invention, the reactor is designed as a tubular reactor. In this embodiment, substrates having an elongated design, in particular coiled cylindrical bodies as indicated in Example 1, are preferably used. These substrates are arranged freely in the tubular reactor or they are bundled in a container. At one end of the tubular reactor, the educt-reaction medium mixture is introduced, while at the other end of the tubular reactor, essentially the product-reaction medium mixture is removed. While the medium is flowing through the tubular reactor, a continuous flow of medium through the supporting body/substrate is taking place. The length of the tubular reactor and the flow rate of the fluid medium and the associated dwell time will be adjusted by those skilled in the art in accordance with the reaction taking place. Those skilled in the art will recognize the fact that the tubular reactor may additionally be equipped with baffles to induce a turbulent flow. As explained above for the continuously operated agitated reactor, flow with the highest possible Re numbers is desirable to minimize the laminar boundary layer and reduce the diffusion pathways. The baffles may optionally be in the special form of the porous supporting body/substrate. As an alternative, additional shaped bodies may also be introduced to serve as baffles.

Those skilled in the art will recognize the fact that in addition to the basic types of rectors described above, modified types of reactors may also be used for the inventive cell culturing methods without going beyond the scope of the present invention.

This invention will now be explained in greater detail below on the basis of the graphic diagrams in individual preferred aspects. These are not intended to restrict the invention to certain forms or arrangements.

The invention will now be further described by way of the following non-limiting examples.

### EXAMPLES

### Example 1

For the intended application as a supporting body/substrate material in the inventive cell culturing process, a polymer composite containing natural fibers and having a weight per unit of area of 100 grams per square meter and a dry layer thickness of 110 micrometers was rolled up to form a body shape having the dimensions: 150 millimeters length and 70 millimeters diameter. Radially closed flow channels with an average channel diameter of 3 millimeters were produced by shaping corrugations from the flat material approximately 8 meters long, and this single layer corrugated structure was then rolled up in the transverse direction and secured in this form. These shaped bodies were carbonized in a nitrogen atmosphere at 800° C. for 48 hours, adding air toward the end to modify the porosity. A weight loss of 61 wt % occurred. The resulting material had a pH of 7.4 in water and a buffer range in the weak acid range. Disks with a diameter of 60 millimeters each and a thickness of 20 millimeters cut from this carbonized material had the following properties:

Surface-to-volume ratio 1700 m²/m³, free flow cross section 0.6 m²/m³; no measurable pressure drop could be detected in flow of water through the material under experimental conditions due to the open structure and flow channel length of 20 millimeters.

These disks were installed in an alternating pressure apparatus according to FIG. 4, so that 500 mL culture medium and 150 mL cell suspension could flow through each disk under sterile conditions. The cell suspension contained cell lines producing hybridoma FLT2 MAB against Shiga toxin, known for non-adherent, non-adhesive growth in suspension.

For comparison purposes, corresponding units were used without a substrate and without carbon material under otherwise the same conditions and same feed rate and/or loading. The liquid medium was passed through the cartridge in a 30-second cycle, i.e., it was circulated, i.e., the body was immersed in the liquid medium every 30 seconds.

The samples with a substrate had a spontaneous quantitative immobilization of cells (the previously cloudy supernatant became clear after about 4 hours) and then no more turbidity of the suspension could be detected. Within an incubation time of seven days, the cell density had increased by a factor of seven to 1.8×10⁷ cells per mL. The MAB production increased from 50 μg/mL at first to 350 μL/mL of the average culture lifetime without any signs of proteolytic degradation. After 25 days, 12 of 12 samples were still viable, after which the process was terminated. This shows that the inventive supporting bodies/substrates lead to an interruption in contact inhibition despite the higher cell density. Even after cryopreservation and thawing, MAB production resumed spontaneously after adding fresh culture medium.

In a comparative experiment, only one of six cultures survived to the 11^{th} day.

### Example 2

### Cross Geometry

For the intended application as a supporting body/substrate material for cell culturing systems, a polymer composite containing natural fibers and having a weight of 100 g/m² and a dry layer thickness of 110 micrometers was shaped into a body having dimensions 300 millimeters length, 150 millimeters width and 50 millimeters height and glued in that form. This produced flow channels having an average channel diameter of 3 millimeters due to corrugation of the flat materials and lamination of these single layer corrugated structures which were then offset by 90° each and had flow channels that were closed radially. These shaped bodies were carbonized at 800° C. for 48 hours in a nitrogen atmosphere, with air being added toward the end to modify the porosity. A weight loss of 61 wt % occurred. The resulting material had a pH of 7.4 in water and a buffering range in weak acids.

Water jet cutting was used to produce cylindrical substrates of this carbonaceous material with dimensions of a diameter of 35 millimeters and a thickness of 40 millimeters, having the following properties:

Surface-to-volume ratio 1700 m²/m³, free flow cross section 0.6 m²/m³; no measurable pressure drop could be detected in flow of water through the supporting body/substrate under experimental conditions due to the open structure and flow channel length of 20 millimeters.

These disks were placed in a radiation crosslinked protective shell and joined to form strands 160 millimeters in length. Each of these strands was inserted into a conventional 2-liter roller bottle and charged with 500 mL liquid culture medium and 150 mL cell suspension under sterile conditions. The cell suspension contained cell lines producing hybridoma FLT2 MAB against Shiga toxin, which is known for non-adherent, non-adhesive growth in suspension.

For comparison purposes, corresponding roller bottles without carbon material were used under otherwise the same conditions and loading.

The roller bottles were rotated on a roller bottle apparatus.

The samples with supporting body/substrate showed a spontaneous quantitative immobilization of cells (the previously cloudy supernatant became clear after approximately four hours) and no more turbidity of the suspension could be detected. Within seven days incubation time, the cell density had increased by a factor of 7 to 1.8×10⁷ cells per mL. MAB production increased from initially 50 μg/mL to 350 μL/mL of the average culture lifetime without any signs of proteolytic degradation. After 25 days, 12 of 12 samples were still viable, after which the experiment was terminated. This shows that the inventive substrates lead to an interruption in contact inhibition despite the higher cell density. Even after cryopreservation and thawing, MAB production resumed spontaneously after adding fresh culture medium.

In the comparative experiment, only one of six cultures survived until day 11.

### Example 3

For the intended application as a supporting body/substrate material for cell culturing systems, a polymer composite containing natural fibers and having a weight of 100 grams per square meter and a dry layer thickness of 110 micrometers was shaped into a body having as dimensions a length of 150 millimeters and a diameter of 70 millimeters was produced by rolling it up. To do so, flow channels with an S shape or a corrugated shape and an average channel diameter of 3 millimeters, previously closed radially, were produced by embossing and then corrugated the flat material and this single layer corrugated structure was then rolled up (see Example 1). These shaped bodies were carbonized at 800° C. for 48 hours in a nitrogen atmosphere, adding air toward the end to modify the porosity. A weight loss of 61 wt % occurred. The resulting material had a pH of 7.4 in water and a buffering range in weak acids.

Disks with a diameter of 60 millimeters and a thickness of 20 millimeters of this carbonaceous material have the following properties:

Surface-to-volume ratio 2500 m²/m³, free flow cross section 0.3 m²/m³; no measurable pressure drop could be detected in flow of water through the supporting body/substrate under experimental conditions due to the open structure and flow channel length of 20 millimeters.

These disks were installed in an apparatus according to FIG. 3 so that 500 mL culture medium and 150 mL cell suspension could flow through each under sterile conditions. The cell suspension contained cell lines producing hybridoma FLT2 MAB against Shiga toxin, known for non-adherent, non-adhesive growth in suspension.

For comparison purposes, corresponding units were used without a substrate and without carbon material under otherwise the same conditions and same feed rate and/or loading.

The liquid medium was passed through the cartridge in a 30-second cycle, i.e., it was circulated, i.e., the body was immersed in the liquid medium every 30 seconds.

The samples with a supporting body/substrate had a spontaneous quantitative immobilization of cells (the previously cloudy supernatant became clear after about four hours) and then no more turbidity of the suspension could be detected. Within an incubation time of seven days, the cell density had increased by a factor of seven to 1.8×10⁷ cells per mL. The MAB production increased from initially 50 μg/mL to 350 μL/mL of the average culture lifetime without any signs of proteolytic degradation. After 25 days, 12 of 12 samples were still viable, after which the process was terminated. This shows that the inventive substrates lead to an interruption in contact inhibition despite the higher cell density. Even after cryopreservation and thawing, MAB production resumed spontaneously after adding fresh culture medium.

### Example 4

The disks from Example 1 were impregnated in an aqueous solution containing 10% polyvinyl pyrrolidone after carbonization and then were dried again. Next the cartridges were installed in an apparatus according to Example 1 and incubated with culture medium and cells. It was observed that the wetting behavior of the cartridges was improved and the cells were immobilized after only two hours (clarifying the previously cloudy supernatant).

### Example 5

The disks from Example 1 were installed in an apparatus according to FIG. 3 comprising two containers which were interconnected by corresponding lines centrally at the bottom.

This container system was incubated with culture medium and cells according to Example 1. The container arrangement was selected so that in the resting position, the carbon disk was still covered with fluid. After waiting for complete immobilization of the cells, the vessel together with the carbon disk was lifted mechanically to the extent that the liquid could escape through the corresponding lines into the second liquid container and the carbon disk was no longer immersed in the liquid. Then the container was lowered back into the resting position. The cycle time for the entire process was 30 seconds. The advantage of this circulation was that the force required to move the media was expended by raising and lowering the cartridges and thus no contact with the media was required.

Within seven days of incubation time, the cell density had increased by a seven to 1.8×10⁷ cells per milliliter. MAB production increased from initially 50 μg/mL to 350 μL/mL of the average culture lifetime without any signs of proteolytic degradation. After 25 days, 12 of 12 samples were still viable, after which the experiment was terminated. This shows that the inventive supporting body/substrates lead to an interruption in contact inhibition despite the higher cell density. Even after cryopreservation and thawing, MAB production resumed spontaneously after adding fresh culture medium.

### Example 6

The disks from Example 1 were installed in an apparatus according to FIG. 3 comprising two containers which were interconnected by corresponding lines at the bottom center.

This container system was incubated with culture medium and cells according to Example 1. The container arrangement was selected so that the carbon disk in the resting position was just covered with fluid. After waiting for complete immobilization of the cells, the container together with the carbon disk was lowered mechanically so that the liquid could flow out of the second liquid container through the corresponding lines and could flow through the carbon disk. Then the container was raised into the resting position again. The cycle time for the entire process was 30 seconds. The advantage of this circulation was that the force required to move the media was expended by raising and/or lowering the cartridges and thus no contact with the media was required.

Within 7 days of incubation time, the cell density had increased by a seven to 1.8×10⁷ cells per milliliter. MAB production increased from initially 50 μg/mL to 350 μL/mL of the average culture lifetime without any signs of proteolytic degradation. After 25 days, 12 of 12 samples were still viable, after which the experiment was terminated. This shows that the inventive supporting bodies/substrates lead to an interruption in contact inhibition despite the higher cell density. Even after cryopreservation and thawing, MAB production resumed spontaneously after adding fresh culture medium.

The invention is further described by the following numbered paragraphs:
1. A method for culturing cells comprising the following steps:
   a) providing a carbon based supporting body having a layered structure, comprising:
      i) at least two porous material layers that are essentially arranged on top of each other, between which a flow-throughable interspace exists; or
      ii) at least one porous material layer that, while keeping its shape, is rolled up in itself or arranged in such a way that a flow-throughable interspace exists between at least two sections of the material layer that are on top of each other; and
   b) loading the supporting body with biological material which is living and/or capable of multiplication;
   c) contacting the loaded supporting body with a fluid medium.
2. The method according to Paragraph 1,
   characterized in that the supporting body comprises a multiplicity of material layers, and that between two material layers each that are arranged on top of each other, at least one interspace exists.
3. The method according to Paragraph 1 or 2,
   characterized in that the interspace between two material layers each or between two sections each of the one rolled up material layer has a multiplicity of channels that run essentially parallel to one another.
4. The method according to Paragraph 3,
   characterized in that the channels that are arranged essentially parallel to one another each have an average channel diameter in the range of about 1 nm to about 1 m, in particular about 1 nm to about 10 cm, preferably 10 nm to 10 mm, and especially preferred 50 nm to 1 mm.
5. The method according to any one of Paragraphs 3 or 4
   characterized in that the channels between a first and a second material layer each are arranged with an angular offset with respect to the channels in an adjacent layer between said second material layer and a third material layer, with an angle of greater than 0° up to 90°, preferably 30 to 90°, and especially preferred 45 to 90°, so that the supporting body exhibits channel layers that are alternatingly angularly offset with respect to one.
6. The method according to any one of the preceding paragraphs,
   characterized in that the channels that run essentially parallel are linear, wave-like, meandering, or zigzag within a layer.
7. The method according to any one of the preceding paragraphs, characterized in that the porous material layer and/or the channel walls have average pore sizes in the range of about 1 nm to 10 cm, preferably 10 nm to 10 mm, and especially preferred 50 nm to 1 mm.
8. The method according to any one of the preceding paragraphs, characterized in that as porous supporting body, a modular structure is used that is produced by carbonization of an optionally structured, rolled, embossed, pre-treated, and/or folded sheet material on the basis of fiber, paper, textile, or polymer material.
9. The method according to any one of the preceding paragraphs, characterized in that the biological material is selected from single-cell or multi-cell microorganisms, fungi, yeasts, spores, plant cells, cell cultures or tissues or animal and/or human cells, cell cultures or tissues, or mixtures thereof.
10. The method according to any one of the preceding paragraphs, characterized in that the loading of the supporting body leads to substantially extensive immobilization of the biological material in and/or on the supporting body.
11. The method according to any one of the preceding paragraphs, characterized in that the medium is selected from liquids or gases, solvents, water, gaseous or liquid or solid reaction educts and/or products, liquid culture media for enzymes, cells and tissues, mixtures thereof and the like.
12. The method according to any one of the preceding paragraphs, characterized in that the supporting body is arranged in a housing, or in or on a suitable container selected from reactors for chemical or biological reactions such as flasks, bottles, especially cell culture bottles, roller bottles, spinner bottles, culture tubes, cell culture chambers, cell culture dishes, culture plates, pipette caps, snap cover glasses, cryotubes, agitated reactors, fixed bed reactors, tubular reactors and the like.
13. The method according to Paragraph 12,
   characterized in that the supporting body is brought in contact with the fluid medium by at least partially filling the container.
14. The method according to Paragraph 13,
   characterized in that the supporting body is moved in the medium in the container.
15. The method according to Paragraph 12 or 13,
   characterized in that the container is connected to a supply vessel filled with the medium by way of feed mechanisms and optionally removal mechanisms are also provided to pass the medium continuously or discontinuously into and through the container.
16. The method according to any one of the preceding paragraphs, characterized in that a fluid medium flows either continuously or discontinuously through the supporting body which is optionally immersed in a container.
17. The method according to Paragraph 16,
   characterized in that the flow of fluid medium through the supporting body is accomplished by moving the supporting body in the medium.
18. The method according to Paragraph 16,
   characterized in that the flow of fluid medium through the supporting body is accomplished by moving the medium in the supporting body.
19. The method according to any one of the preceding paragraphs, characterized in that nutrients are provided with the medium and/or metabolites are removed with the medium either continuously or discontinuously.
20. A porous carbon-based supporting body as described in one of the preceding paragraphs, comprising immobilized biological material which is living and/or capable of multiplication.
21. The supporting body according to Paragraph 20,
   characterized in that the biological material is selected from single-cell or multi-cell microorganisms, yeasts, fungi, spores, plant cells, cells cultures or tissues or animal and/or human cells, cell cultures or tissue or mixtures thereof.
22. The supporting body according to Paragraph 20 or 21, comprised of activated carbon, sintered activated carbon, amorphous, crystalline or partially crystalline carbon, graphite, pyrolytic carbonaceous material, carbon fibers or carbides, carbonitrides, oxycarbides and/or oxycarbonitrides of metals or nonmetals as well as mixtures thereof.
23. The supporting body according to any one of Paragraphs 20 through 22, characterized in that it contains between 10⁻⁵ wt % and 99 wt %, preferably between 10⁻² wt % and 80 wt %, most preferably between 1 wt % and 50 wt % cells, based on the total weight of the loaded supporting body.
24. A reactor for culturing cells, comprising one or more supporting bodys according to Paragraphs 20 through 23.
25. The reactor according to Paragraph 24, selected from reactors for chemical or biological reactions such as flasks, bottles, especially cell culture flasks, roller bottles, spinner bottles, culture tubes, cell culture chambers, cell culture dishes, culture plates, pipette caps, snap cover glasses, cryotubes, agitated reactors, fixed bed reactors and tubular reactors.
26. A roller bottle comprising a supporting body according to any one of Paragraphs 20 through 23.
27. A cartridge comprising a supporting body according to any one of Paragraphs 20 through 26 in a housing.

Having thus described in detail preferred embodiments of the present invention, it is to be understood that the invention defined by the above paragraphs is not to be limited to particular details set forth in the above description as many apparent variations thereof are possible without departing from the spirit or scope of the present invention.

What is claimed is:

1. A method for culturing cells comprising the following steps:
a) providing a carbon based supporting body comprised of a material selected from activated carbon, sintered activated carbon, amorphous, crystalline or partially crystalline carbon, graphite, pyrolytic carbonaceous material, carbon fibers or carbides, carbonitrides, oxycarbides and/or oxycarbonitrides of metals or nonmetals as well as mixtures of these materials, the supporting body having a layered structure, comprising:
i) at least two porous material layers that are essentially arranged on top of each other, between which a flow-throughable interspace exists; or
ii) at least one porous material layer that, while keeping its shape, is rolled up in itself or arranged in such a way that a flow-throughable interspace exists between at least two sections of the material layer that are on top of each other; and
iii) the interspace between two material layers each or between two sections each of the rolled up material layer has a multiplicity of channels that run essentially parallel to one another;
b) loading the supporting body with biological material which is living and/or capable of multiplication;
c) contacting the loaded supporting body with a fluid medium.

2. The method according to claim 1,
characterized in that the supporting body comprises a multiplicity of material layers, and that between two material layers each that are arranged on top of each other, at least one interspace exists.

3. The method according to claim 1,
characterized in that the channels that are arranged essentially parallel to one another each have an average channel diameter in the range of about 1 nm to about 1 m, in particular about 1 nm to about 10 cm, preferably 10 nm to 10 mm, and especially preferred 50 nm to 1 mm.

4. The method according to claim 1,
characterized in that the channels between a first and a second material layer each are arranged with an angular offset with respect to the channels in an adjacent layer between said second material layer and a third material layer, with an angle of greater than 0° up to 90°, preferably 30 to 90°, and especially preferred 45 to 90°, so that the supporting body exhibits channel layers that are alternatingly angularly offset with respect to one.

5. The method according to claim 1,
characterized in that the channels that run essentially parallel are linear, wave-like, meandering, or zigzag within a layer.

6. The method according to claim 1, characterized in that the porous material layer and/or the channel walls have average pore sizes in the range of about 1 nm to 10 cm, preferably 10 nm to 10 mm, and especially preferred 50 nm to 1 mm.

7. The method according to claim 1, characterized in that as porous supporting body, a modular structure is used that is produced by carbonization of an optionally structured, rolled, embossed, pre-treated, and/or folded sheet material on the basis of fiber, paper, textile, or polymer material.

8. The method according to claim 1, characterized in that the biological material is selected from single-cell or multi-cell microorganisms, fungi, yeasts, spores, plant cells, cell cultures or tissues or animal and/or human cells, cell cultures or tissues, or mixtures thereof.

9. The method according to claim 1, characterized in that the loading of the supporting body leads to substantially extensive immobilization of the biological material in and/or on the supporting body.

10. The method according to claim 1, characterized in that the medium is selected from liquids or gases, solvents, water, gaseous or liquid or solid reaction educts and/or products, liquid culture media for enzymes, cells and tissues, mixtures thereof and the like.

11. The method according to claim 1, characterized in that the supporting body is arranged in a housing, or in or on a suitable container selected from reactors for chemical or biological reactions such as flasks, bottles, especially cell culture bottles, roller bottles, spinner bottles, culture tubes, cell culture chambers, cell culture dishes, culture plates, pipette caps, snap cover glasses, cryotubes, agitated reactors, fixed bed reactors, tubular reactors and the like.

12. The method according to claim 11,
characterized in that the supporting body is brought in contact with the fluid medium by at least partially filling the container.

13. The method according to claim 12,
characterized in that the supporting body is moved in the medium in the container.

14. The method according to claim 11,
characterized in that the container is connected to a supply vessel filled with the medium by way of feed mechanisms and optionally removal mechanisms are also provided to pass the medium continuously or discontinuously into and through the container.

15. The method according to claim 1, characterized in that a fluid medium flows either continuously or discontinuously through the supporting body which is optionally immersed in a container.

16. The method according to claim 15,
characterized in that the flow of fluid medium through the supporting body is accomplished by moving the supporting body in the medium.

17. The method according to claim 15,
characterized in that the flow of fluid medium through the supporting body is accomplished by moving the medium in the supporting body.

18. The method according to claim 1, characterized in that nutrients are provided with the medium and/or metabolites are removed with the medium either continuously or discontinuously.

19. A porous carbon-based supporting body, comprised of a material selected from activated carbon, sintered activated carbon, amorphous, crystalline or partially crystalline carbon, graphite, pyrolytic carbonaceous material, carbon fibers or carbides, carbonitrides, oxycarbides and/or oxycarbonitrides of metals or nonmetals as well as mixtures of these materials, the supporting body having a layered structure, comprising:
i) at least two porous material layers that are essentially arranged on top of each other, between which a flow-throughable interspace exists; or
ii) at least one porous material layer that, while keeping its shape, is rolled up in itself or arranged in such a way that a flow-throughable interspace exists between at least two sections of the material layer that are on top of each other; and
iii) the interspace between two material layers each or between two sections each of the rolled up material layer has a multiplicity of channels that run essentially parallel to one another;
comprising immobilized biological material which is living and/or capable of multiplication.

20. The supporting body according to claim 19,
characterized in that the biological material is selected from single-cell or multi-cell microorganisms, yeasts, fungi, spores, plant cells, cells cultures or tissues or animal and/or human cells, cell cultures or tissue or mixtures thereof.

21. The supporting body according to claim 19,
characterized in that it contains between 10⁻⁵ wt % and 99 wt %, preferably between 10⁻² wt % and 80 wt %, most preferably between 1 wt % and 50 wt % cells, based on the total weight of the loaded supporting body.

22. A reactor for culturing cells, comprising one or more supporting bodies according to claims 19.

23. The reactor according to claim 22, selected from reactors for chemical or biological reactions such as flasks, bottles, especially cell culture flasks, roller bottles, spinner bottles, culture tubes, cell culture chambers, cell culture dishes, culture plates, pipette caps, snap cover glasses, cryotubes, agitated reactors, fixed bed reactors and tubular reactors.

24. A roller bottle comprising a supporting body according to claim 19.

25. A cartridge comprising a supporting body according to claim 19.
